(19) United States
(12) Patent Application Publication
(10) Pub. No.: US 2011/0045524 A1
(43) Pub. Date: Feb. 24, 2011

(54) TRANSFECTION READY EUKARYOTIC CELLS

(75) Inventors: Marie Callahan, San Diego, CA (US); Michael Lee, San Diego, CA (US); Alan Greener, San Diego, CA (US); Anthony Sorge, Rancho Santa Fe, CA (US)

Correspondence Address: KNOBBE MARTENS OLSON & BEAR LLP, 2040 MAIN STREET, FOURTEENTH FLOOR, IRVINE, CA, 92614, US

(73) Assignee: GENE THERAPY SYSTEMS, INC., SAN DIEGO, CA (US)

(21) Appl. No.: 12/861,112

(22) Filed: Aug. 23, 2010

Related U.S. Application Data
(62) Division of application No. 12/049,194, filed on Mar. 14, 2008, now abandoned.
(60) Provisional application No. 60/895,430, filed on Mar. 16, 2007.

(51) Int. Cl. C12Q 1/02; C12N 15/87

(52) U.S. Cl. 435/29; 435/455

(57) ABSTRACT

Described herein are frozen populations of transfection ready competent eukaryotic cells, transfection kits comprising the frozen populations of transfection ready competent cells, and methods of using the same.

### RELATED APPLICATIONS

The present application is a divisional of and claims priority under 35 U.S.C. §121 to U.S. application Ser. No. 12/049,194, entitled “TRANSFECTION READY EUKARYOTIC CELLS,” filed Mar. 14, 2008, to Callahan et al, which claims priority to U.S. Provisional Application Ser. No. 60/895,430, filed on Mar. 16, 2007, by Callahan et al., entitled “TRANSFECTION READY EUKARYOTIC CELLS,” which is hereby expressly incorporated by reference in its entirety.

### BACKGROUND OF THE INVENTION

1. Field of the Invention

The embodiments disclosed herein relate generally to the field of molecular biology. In particular, the embodiments provided herein relate to transfection-ready eukaryotic cells, kits containing transfection ready eukaryotic cells, and methods of making and using the transfection-ready eukaryotic cells and kits.

2. Description of the Related Art

The advent of recombinant nucleic acid technology and genetic engineering has led to numerous efforts to develop methods that facilitate the transfection of macromolecules into eukaryotic cells. Transfection of eukaryotic cells is a term used to describe the introduction of foreign material into the cell, to allow uptake of the material. Most commonly, transfection refers to the uptake of nucleic acids or nucleic acid analogs such as DNA, RNA, siRNA, cDNA, however, the term can also refer to the uptake of proteins or other macromolecules.

One challenge with existing transfection techniques is that certain types of cells are more difficult to efficiently transfect. Another challenge is that significant time is required to prepare cells that are ready for transfection, for example, cells may require passage for one or more days before they can be transfected. Also, the passaged cells can differentiate with each passage so that they will have variable characteristics and transfection efficiencies. Furthermore, for many cell lines, even those that are considered easy to transfect, there are limitations on the cell density that one can transfect efficiently.

In view of the foregoing, there is a need for a method of transfecting cells which results in high transfection efficiencies, with higher cell densities, with a wide variety of mammalian host cell lines, and which does not require continuous culturing of cells which can be both time consuming and result in variability in experimental results.

### SUMMARY OF THE INVENTION

Provided herein are compositions and kits that include transfection ready eukaryotic cells, as well as methods of making the cells, compositions and kits. Also provided are methods of using the same, for example, methods of transfecting of eukaryotic cells and methods of screening nucleic acid expression constructs.

Provided herein are methods of making a transfection competent eukaryotic cells. The method can include the steps of culturing a population of eukaryotic cells in a first culture medium supplemented with about 10% serum. The cells can be cultured until the culture reaches about 80% to about 100% confluency. The cells can be harvested and diluted to a density of about 1×10⁴ cells/mL to about 1×10⁸ cells/mL, preferably to a density of about 1×10⁵ cells/mL to about 5×10⁷ cells/mL, and most preferably to a density of about 5×10⁶ cells/mL in a second medium that includes a culture medium supplemented with about 20% to about 40% (v/v) serum. The second culture medium can also include about 5% to about 20% cryoprotectant (v/v).

Also, the cells can be aliquoted into 10 or more, 50 or more, 100 or more, or 1000 or more aliquots. The aliquots can be placed into an appropriate containment device, for example a vial. The aliquots can be frozen.

Preferably, the second culture medium includes about 30% serum (v/v), and about 10% (v/v) cryoprotectant. In some embodiments, the cryoprotectant can be dimethyl sulfoxide (DMSO), glycerol, 1,2-propanediol, or any combination thereof. Preferably, the cryoprotectant is DMSO.

Optionally, the second culture medium can include at least one compound selected from the group consisting of a sugar or a sugar alcohol at a concentration of about 5% to about 20% (w/v), preferably at a concentration of about 10% (w/v). In some embodiments, the second culture medium is supplemented with trehalose, for example at a concentration of about 10%.

In some embodiments the eukaryotic cells are mammalian cells. For example, in some embodiments the eukaryotic cells can be CHO cells, HEK293 cells, COS-7 cells, HeLa cells, NIH3T3 cells, MCF-7 cells, Jurkat cells or RAW264 cells.

In some embodiments, the first culture medium and said second culture medium are Dulbecco's Modified Eagle's Medium (DMEM). In some embodiments, the first culture medium and said second culture medium can include supplements, such as non-essential amino acids (NEAA). Preferably, NEAA is provided at a concentration of about 1% (v/v).

Other embodiments relate to a frozen population of eukaryotic cells. The frozen eukaryotic cells can be present at a concentration of about 1×10⁴ cells/mL to about 1×10⁸ cells/mL, preferably at about 1×10⁵ cells/mL to about 5×10⁷ cells/mL, and most preferably at about 5×10⁶ cells/mL in a culture medium supplemented with about 20% to about 40%, preferably about 25% to about 35%, e.g., 30% serum and a cryoprotectant. In some embodiments, the cryoprotectant is present in a concentration of about 5-30%. In some embodiments, the culture medium in which the cells are frozen is supplemented with at least one compound selected from the group consisting of sugars and sugar alcohols at a concentration of about 5% to about 20% (w/v), preferably at a concentration of about 10% (w/v). In some embodiments, the culture medium includes trehalose, for example 10% trehalose. Preferably, the culture medium is DMEM supplemented with NEAA, for example DMEM with 1% NEAA.

In some embodiments, the eukaryotic cells are mammalian cells, such as CHO cells, HEK293 cells, COS-7 cells, HeLa cells, NIH3T3 cells, MCF-7 cells, Jurkat cells or RAW264 cells.

Other embodiments provide a eukaryotic cell transfection kit. In some embodiments, the transfection kit includes a frozen population of eukaryotic cells at a concentration of about 1×10⁴ cells/mL to about 1×10⁸ cells/mL, preferably about 1×10⁵ cells/mL to about 5×10⁷ cells/mL, and most preferably at about 5×10⁶ cells/mL. The frozen population of eukaryotic cells can be present in a culture medium supplemented with about 30% serum and a cryoprotectant, wherein said cryoprotectant is present in a concentration of about 5%-30%.

Preferably, the eukaryotic cells are mammalian cells such as CHO cells, HEK293 cells, COS-7 cells, HeLa cells, NIH3T3 cells, MCF-7 cells, Jurkat cells or RAW264 cells. In some embodiments, the kits can include instructions, for example instructions for transfecting the cells.

Also provided are methods of screening eukaryotic cells for a detectable signal encoded on a nucleic acid. In some embodiments, a population of frozen eukaryotic cells is provided and thawed, and placed in a containment device. The thawed cells can be transfected with the nucleic acid within about five hours, and preferably within about three hours, of placing them in the containment device. The detectable signal from the eukaryotic cells can be measured.

In preferred embodiments, the eukaryotic cells are mammalian cells, such as CHO cells, HEK293 cells, COS-7 cells, HeLa cells, NIH3T3 cells, MCF-7 cells, Jurkat cells or RAW264 cells.

Yet other embodiments relate to a vial comprising frozen eukaryotic cells. The cells can be produced by a method including the steps of culturing a population of eukaryotic cells in a first culture medium supplemented with about 10% serum. The cells can be cultured until the culture reaches about 80% to about 100% confluency. The cells can be harvested and diluted to a density of about 1×10⁴ cells/mL to about 1×10⁸ cells/mL, preferably to a density of about 1×10⁵ cells/mL to about 5×10⁷ cells/mL, and most preferably to a density of about 5×10⁶ cells/mL in a second medium that includes a culture medium supplemented with about 20% to about 40% (v/v) serum. The second culture medium can also include about 5% to about 20% cryoprotectant (v/v). The cells can be aliquoted into a vial and subsequently frozen.

Preferably, the second culture medium includes about 30% (v/v) serum, and about 10% (v/v) cryoprotectant. In some embodiments, the cryoprotectant can be dimethyl sulfoxide (DMSO), glycerol, 1,2-propanediol, or any combination thereof. Preferably, the cryoprotectant is DMSO.

Optionally, the second culture medium can include at least one compound selected from the group consisting of a sugar or a sugar alcohol at a concentration of about 5% to about 20% (w/v), preferably at a concentration of about 10% (w/v). In some embodiments, the second culture medium is supplemented with trehalose, for example at a concentration of about 10%.

In some embodiments the eukaryotic cells are mammalian cells. For example, in some embodiments the eukaryotic cells can be CHO cells, HEK293 cells, COS-7 cells, HeLa cells, NIH3T3 cells, MCF-7 cells, Jurkat cells or RAW264 cells.

In some embodiments, the first culture medium and said second culture medium are Dulbecco's Modified Eagle's Medium (DMEM). In some embodiments, the first culture medium and said second culture medium can include supplements, such as non-essential amino acids (NEAA). Preferably, NEAA is provided at a concentration of about 1% (v/v).

Still other embodiments relate to methods of transfecting a eukaryotic cell. In some embodiments, a frozen eukaryotic cell is provided and thawed. The cell can be contacted with a nucleic acid molecule of interest within less than about five hours, and preferably within about three hours, from when the frozen cells are provided. The nucleic acid can thereby be introduced said nucleic acid molecule of interest into said eukaryotic cell.

In some embodiments, the eukaryotic cell can be a mammalian cell, such as a CHO cell, a HEK293 cell, a COS-7 cell, a HeLa cell, an NIH3T3 cell, an MCF-7 cell, a Jurkat cell or a RAW264 cell.

Optionally, the thawed cell can be contacted with culture medium prior to contacting said cell with said nucleic acid molecule. In some embodiments, the thawed cell can be contacted with culture medium in the presence of a plurality of thawed eukaryotic cells in a density of about 1×10⁴ to about 1×10⁶ cells/mL, for example at about 1.2×10⁵ to about 4×10⁵ cells/mL. e.g., with a plurality of cells at a density of about 3.5×10⁵ cells/mL to about 4×10⁵ cells/mL.

In some embodiments, the eukaryotic cell has been frozen in culture medium supplemented with about 10% to about 40% (v/v) serum, for example in culture medium supplemented with about 30% (v/v) serum.

In some embodiments, the eukaryotic cell has been frozen in culture medium supplemented with a cryoprotectant in a concentration of about 10-30% (v/v), for example 10% (v/v) cryoprotectant. In some embodiments, the cryoprotectant can be dimethyl sulfoxide (DMSO), glycerol, 1,2-propanediol, or any combination thereof. Preferably, the cell has been frozen in culture medium supplemented with DMSO, e.g., 10% DMSO.

In some embodiments, the eukaryotic cell has been frozen in a culture medium supplemented with at least one sugar or sugar alcohols. In some embodiments, the sugar or sugar alcohol can be present at a concentration of about 5% to about 20% (w/v), preferably at a concentration of about 10% (w/v). Preferably, the eukaryotic cell has been frozen in a culture medium supplemented with trehalose, such as 10% (w/v) trehalose.

Other embodiments relate to methods of increasing the transfection efficiency of a eukaryotic cell. In some embodiments, a frozen eukaryotic cell is provided and thawed. The cell can be contacted with a nucleic acid molecule of interest within less than about five hours, and preferably within about three hours, from when the frozen cells are provided. The nucleic acid can thereby be introduced into the eukaryotic cell.

In some embodiments, the eukaryotic cell can be a mammalian cell, such as a CHO cell, a HEK293 cell, a COS-7 cell, a HeLa cell, an NIH3T3 cell, an MCF-7 cell, a Jurkat cell or a RAW264 cell.

Optionally, the thawed cell can be contacted with culture medium prior to contacting said cell with said nucleic acid molecule. In some embodiments, the thawed cell can be contacted with culture medium in the presence of a plurality of thawed eukaryotic cells in a density of about 1.2×10⁵ to about 4×10⁵ cells/mL, e.g., with a plurality of cells at a density of about 3.5×10⁵ cells/mL to about 4×10⁵ cells/mL.

In some embodiments, the eukaryotic cell has been frozen in culture medium supplemented with about 10% to about 40% (v/v) serum, for example in culture medium supplemented with about 30% (v/v) serum.

In some embodiments, the eukaryotic cell has been frozen in culture medium supplemented with a cryoprotectant in a concentration of about 10-30% (v/v), for example 10% (v/v) cryoprotectant. In some embodiments, the cryoprotectant can be dimethyl sulfoxide (DMSO), glycerol, 1,2-propanediol, or any combination thereof. Preferably, the cell has been frozen in culture medium supplemented with DMSO, e.g., 10% DMSO.

In some embodiments, the eukaryotic cell has been frozen in a culture medium supplemented with at least one sugar or sugar alcohols. In some embodiments, the sugar or sugar alcohol can be present at a concentration of about 5% to about 20% (w/v), preferably at a concentration of about 10% (w/v). Preferably, the eukaryotic cell has been frozen in a culture medium supplemented with trehalose, such as 10% (w/v) trehalose.

Also provided are improved methods of transfecting a population of eukaryotic cells, which can eliminate the need to passage the population of eukaryotic cells to confluency just prior to transfection. A frozen eukaryotic cell can be provided and thawed. The cell can be contacted with a nucleic acid molecule of interest within less than about five hours, and preferably within about three hours, from when the frozen cells are provided. The nucleic acid can thereby be introduced into said eukaryotic cell.

In some embodiments, the eukaryotic cell can be a mammalian cell, such as a CHO cell, a HEK293 cell, a COS-7 cell, a HeLa cell, an NIH3T3 cell, an MCF-7 cell, a Jurkat cell or a RAW264 cell.

Optionally, the thawed cell can be contacted with culture medium prior to contacting said cell with said nucleic acid molecule. In some embodiments, the thawed cell can be contacted with culture medium in the presence of a plurality of thawed eukaryotic cells in a density of about 1×10⁴ to about 1×10⁶ cells/mL, for example at about 1.2×10⁵ to about 4×10⁵ cells/mL. e.g., with a plurality of cells at a density of about 3.5×10⁵ cells/mL to about 4×10⁵ cells/mL.

In some embodiments, the eukaryotic cell has been frozen in culture medium supplemented with about 10% to about 40% (v/v) serum, for example in culture medium supplemented with about 30% (v/v) serum.

In some embodiments, the eukaryotic cell has been frozen in culture medium supplemented with a cryoprotectant in a concentration of about 10-30% (v/v), for example 10% (v/v) cryoprotectant. In some embodiments, the cryoprotectant can be dimethyl sulfoxide (DMSO), glycerol, 1,2-propanediol, or any combination thereof. Preferably, the cell has been frozen in culture medium supplemented with DMSO, e.g., 10% DMSO.

In some embodiments, the eukaryotic cell has been frozen in a culture medium supplemented with at least one sugar or sugar alcohols. In some embodiments, the sugar or sugar alcohol can be present at a concentration of about 5% to about 20% (w/v), preferably at a concentration of about 10% (w/v). Preferably, the eukaryotic cell has been frozen in a culture medium supplemented with trehalose, such as 10% (w/v) trehalose.

Yet other methods relate to improved methods of transfecting a population of eukaryotic cells, which allow for an increased density of cells in a transfection reaction. A population of frozen eukaryotic cells is provided and thawed. The thawed population of eukaryotic cells can be contacted with culture medium, such that the thawed cells are present at a density of about 1.2×10⁵ to about 4×10⁵ cells/mL; and contacting said eukaryotic cell population with a nucleic acid molecule of interest within less than about five hours, and preferably within about three hours of after providing the cells.

In some embodiments, the eukaryotic cells can be mammalian cells, such as CHO cells, HEK293 cells, COS-7 cells, HeLa cells, NIH3T3 cells, MCF-7 cells, Jurkat cells or RAW264 cells.

In some embodiments, the eukaryotic cells have been frozen in culture medium supplemented with about 10% to about 40% (v/v) serum, e.g., in a culture medium supplemented with about 30% (v/v) serum.

In some embodiments, the eukaryotic cells have been frozen in culture medium supplemented with a cryoprotectant in a concentration of about 10-30% (v/v), preferably about 10%. For example, the eukaryotic cells can be frozen in a culture medium supplemented with one of the following cryoprotectants; dimethyl sulfoxide (DMSO), glycerol, 1,2-propanediol, or any combination thereof. Preferably, the eukaryotic cell has been frozen in a medium supplemented with DMSO, for example, a medium supplemented with 10% DMSO.

In some embodiments, the eukaryotic cells have been frozen in a culture media supplemented with sugars and/or sugar alcohols at a concentration of about 5% to about 20% (w/v), preferably about 10%. For example, in some embodiments the eukaryotic cells have been frozen in medium supplemented with trehalose, for example 10% trehalose.

Also provided are methods of increasing the number of eukaryotic cells transfected in a transfection reaction. A population of frozen eukaryotic cells can be provided and thawed. The thawed cells can be contacted with culture media such that the thawed cells are present at a density of about 1.0×10⁴ to about 1.0×10⁶, preferably between about 1.0×10⁵ to about 4×10⁵ cells/mL; most preferably at about 2.5×10⁵. The thawed cells can be contacted with a nucleic acid molecule of interest within less than about five hours, and preferably less than about three hours, of said providing the frozen cell population, thereby transfecting the eukaryotic cell population.

In some embodiments, the eukaryotic cells can be mammalian cells, such as CHO cells, HEK293 cells, COS-7 cells, HeLa cells, NIH3T3 cells, MCF-7 cells, Jurkat cells or RAW264 cells.

In some embodiments, the eukaryotic cells have been frozen in culture medium supplemented with about 10% to about 40% (v/v) serum, e.g., in a culture medium supplemented with about 30% (v/v) serum.

In some embodiments, the eukaryotic cells have been frozen in culture medium supplemented with a cryoprotectant in a concentration of about 10-30% (v/v), preferably about 10%. For example, the eukaryotic cells can be frozen in a culture medium supplemented with one of the following cryoprotectants; dimethyl sulfoxide (DMSO), glycerol, 1,2-propanediol, or any combination thereof. Preferably, the eukaryotic cell has been frozen in a medium supplemented with DMSO, for example, a medium supplemented with 10% DMSO.

In some embodiments, the eukaryotic cells have been frozen in a culture media supplemented with sugars and/or sugar alcohols at a concentration of about 5% to about 20% (w/v), preferably about 10%. For example, in some embodiments the eukaryotic cells have been frozen in medium supplemented with trehalose, for example 10% trehalose.

In some embodiments, the transfected cells produce a detectable signal. Thus, in some embodiments, the detectable signal is measured.

### BRIEF DESCRIPTION OF THE DRAWINGS

FIGS. 1A-1F are images of passaged HeLa (FIG. 1A), HEK 293 (FIG. 1C), and CHO-K1 (FIG. 1E) cells and thawed frozen competent HeLa (FIG. 1B), HEK 293 (FIG. 1D), and CHO-K1 (FIG. 1F) cells grown in culture for 48 hours. The images depict the view from under a light microscope.

FIGS. 2A-2F are images of passaged HeLa (FIG. 1A), HEK 293 (FIG. 1C), and CHO-K1 (FIG. 1E) cells and thawed frozen competent HeLa (FIG. 1B), HEK 293 (FIG. 1D), and CHO-K1 (FIG. 1F) cells grown in a 24 well plate, viewed under a light microscope.

FIG. 3 is a bar graph showing the level of β-galactosidase expression in CHO-K1, HEK-293 and HeLa frozen competent cells transfected with a β-galactosidase expression vector compared to CHO-K1, HEK-293 and HeLa cells grown in continuous culture (passaged) cells transfected with the same expression vector. β-galactosidase expression levels are reported as a percentage of the β-galactosidase expression in the passaged cells.

FIG. 4 is a graph showing the β-galactosidase activity in HEK 293 cells transfected with 0.1, 0.25, 0.5, 0.75 or 1 μg of a β-galactosidase expression vector. The closed diamonds represent the activity in HEK 293 cells that were passaged prior to transfection. The closed squares represent the activity in HEK 293 cells that were frozen in media supplemented with 10% DMSO and 30% FBS prior to transfection. The closed triangles represent the activity in HEK 293 cells that were frozen in media supplemented with 10% DMSO, 30% FBS, and 10% trehalose prior to transfection.

FIG. 5 is a bar graph showing the transfection efficiency based on fluorescence activated cell sorting (FACS) analysis of HEK 293 cells transfected with 0.5 μg of a β-galactosidase expression vector. The 293 P label refers to HEK 293 cells that were passaged prior to transfection. The 293 S label refers to cells that were frozen in media supplemented with 10% DMSO and 30% FBS prior to transfection. The 293 T label refers to cells that were frozen in media supplemented with 10% DMSO, 30% FBS, and 10% trehalose prior to transfection. The DNA only label refers to control cells that were contacted with the β-galactosidase expression vector, but which were not contacted with LIPOFECTAMINE™ transfection reagent during the transfection procedure. The GP2 only label refers to control cells that were contacted with LIPOFECTAMINE™ transfection reagent but not the β-galactosidase expression vector during the transfection procedure. The cells only label refers to control cells that were not contacted with either β-galactosidase expression vector or the LIPOFECTAMINE™ transfection reagent during the transfection procedure.

### DETAILED DESCRIPTION OF THE PREFERRED EMBODIMENT

Disclosed herein are improvements in the transfection of eukaryotic cells, including for example, transfection with nucleic acids. Generally, embodiments disclosed herein relate to compositions and kits that comprise transfection ready eukaryotic cells, methods of making the cells, compositions and kits, as well as methods of using the same, for example, methods of transfecting of eukaryotic cells and methods of screening nucleic acid expression constructs.

Transfection of eukaryotic cells can involve the transient disruption of the cell wall. A number of protocols for performing transfections have been developed, many of which involve the use of either calcium phosphate or DEAE-dextran (or its analogs) as a carrier to promote the uptake of exogenous nucleic acids by cultured eukaryotic cells, such as mammalian cells. Other methods have used “lipofection” techniques, which incorporate the use of synthetic cationic lipids to effect the transfection. Osmotic shock of the cells or treatment of the cells with lysosomal inhibitors has been used in an attempt to enhance transfection efficiencies. Other attempts to increase the efficiencies have used high-voltage electric pulses to create pores in the cell membranes to increase the efficiency of nucleic acid uptake by the cells.

In reference to transfection efficiency, using previous transfection methods cell lines can be characterized as easy to transfect, more difficult to transfect, and very hard to transfect. Non-limiting examples of easy to transfect mammalian cells include CHO-K1 cells, HEK293 cells and COS-7 cells, which can exhibit transfection efficiencies greater than 95%. Non-limiting examples of mammalian cells that are more difficult to transfect include HeLa cells, NIH3T3 cells, and MCF-7 cells, which can exhibit transfection efficiencies ranging from 10%-50%. Non-limiting examples of mammalian cells that are very hard to transfect and that exhibit transfection efficiencies lower than 10% include Jurkat cells and RAW264 cells.

A problem that can exist for any transfected cell, regardless of its ease of transfection, is the issue of decreased viability of the transfected cell population. As such, existing eukaryotic transfection methods can require a certain density of cells for optimal transfection. Also, previous transfection methods can require the presence of a sufficient number of cells in an appropriate stage of growth in order to obtain a sufficient number of transfected cells. This generally has required cell cultures to be grown and passaged until the appropriate cell density is reached, which may take more than 48 hours. The continuous culturing of cell lines used in transfection experiments may also lead to experimental variability.

Achieving a high number of viable cells transfected with a nucleic acid of interest can be particularly important in the context of high throughput screening or small scale testing of expression constructs. Specifically, it can be desirable to maximize the protein yield per cell culture vessel to enable enhanced assay sensitivity. Screening assays such as high throughput screens are often limited to cell lines that have high cell transfection efficiencies. Thus, it is desirable to improve the transfection efficiency of cell lines that are typically more difficult to transfect, to overcome the limitations on the choices of cell lines used in screening assays.

The cells, compositions, kits, and methods of making and using the same disclosed herein alleviate or overcome many of the above-mentioned challenges related to transfection. In particular, the embodiments disclosed herein provide improvements in one or more of the areas related to transfection efficiency of cell lines, the cell density of transfected cells, speed and timing for performing transfection due to providing transfection ready cells, and improved sensitivity in assays and screens that utilize transfected cells.

### Transfection Ready Eukaryotic Cells

Some embodiments relate to methods of making transfection competent eukaryotic cells, including preferably, frozen, competent eukaryotic cells. Generally, the methods involve culturing the eukaryotic cells until about they reach the desired confluency, and harvesting the cells and diluting them in culture medium supplemented with serum and a cryoprotectant.

Any eukaryotic cell can be used. The eukaryotic cells and cell populations included in the embodiments described herein can be yeast cells, insect cells, plant cells, or various animal cells, such as mammalian cells. Non-limiting examples of yeast cells that can be used in the embodiments disclosed herein include Saccharomyces cerevisiae, Pichia pastoris or Schizosaccharomycetes pombe cells. Non-limiting examples of insect cells that can be used in the embodiments disclosed herein include Sf9 cells, S2 cells, Lymantria dispar cells, or the like. Non-limiting examples of plant cells that can be used in the embodiments disclosed herein include Arabidopsis, Nicotina tabacam and the like.

In preferred embodiments, the eukaryotic cells are mammalian cells. Eukaryotic cell lines including mammalian cell lines can be obtained from commercial or other publicly available sources such as the ATCC. Non-limiting examples of mammalian cells useful in the embodiments disclosed herein include CHO cells, COS-7 cells, NIH-3T3 cells, HEK 293 cells, HeLa cells, MCF-7 cells, Jurkat cells and RAW 264 cells, and the like.

The eukaryotic cells can be cultured in a culture vessel in a first cell culture media. Culture vessels can include standard, commercially available tissue culture dishes, vials, flasks and other “glassware” (or containers made of other materials such as plastics, etc.), multiwell cell culture plates such as 24-well, 96 well, 384 well, and 1536 well plates, or the like.

The cell cultures can be grown in culture media supplemented with serum until the cells reach about 90% to about 100% confluency. The cells can be cultured in any standard culture media, depending on the type of eukaryotic cell. For example, culture media useful for the culturing of mammalian cells includes Dulbecco's Modified Eagle's Medium (DMEM), Minimum Essential Media Eagle (MEM), Medium 199, Ames' Media, BGJb Medium (Fitton-Jackson Modification), Click's Medium, CMRL-1066 Medium, Fischer's Medium, Glascow Minimum Essential Medium (GMEM), Iscove's Modified Dulbecco's Medium (IMDM), L-15 Medium (Leibovitz), McCoy's 5A Modified Medium, NCTC Medium, Swim's S-77 Medium, Waymouth Medium, William's Medium E, RPMI Medium, and the like. Preferably, mammalian cells are cultured in DMEM Medium.

In some embodiments, the medium can be supplemented with nutrients such as amino acids. For example, in some embodiments, the first culture media can comprises DMEM supplemented with non-essential amino acids (NEAA) and the like. Preferably, the DMEM is supplemented with about 1% (v/v) NEAA.

The serum can be derived from calves, e.g., fetal bovine serum, adult cows, newborn cows, horses, pigs, rabbits, goats, or any other mammal. Sera useful in the embodiments disclosed herein can be purchased from a variety of sources. Preferably, fetal bovine serum is used to supplement mammalian cell culture media. The serum supplement can be added to a concentration of at least about 1% to about 20% or 30% (v/v), for example at 1%, 2,%, 3%, 4%, 5%, 6%, 7%, 8% 9%, 10%, 11%, 12%, 13%, 14%, 15%, 16%, 17%, 18%, 19%, 20% (v/v) or more. Preferably, the first media is supplemented with about 10% fetal bovine serum. In some embodiments, sera from non-animal sources can be used.

The cell cultures can be examined using standard protocols to assess the confluency. When the cells have reached about 90 to about 100% confluency, the cells can be harvested. In some embodiments, the eukaryotic cells grow in suspension. These cells can be directly harvested using standard protocols. See, e.g., J. Bonafaciano, et al. Ed, Current Protocols in Cell Biology, © 2007, John Wiley and Sons (Hoboken, N.J.), which is incorporated herein by reference in its entirety, for the mentioned protocols and all others. In some embodiments, the eukaryotic cells can be adherent. Depending on whether the eukaryotic cells are adherent or grow in suspension, the cells can be treated to detach them from the culture vessel prior to harvesting the cells. Detaching the eukaryotic cells can be accomplished using standard methods, such as scraping, and enzymatic treatment such as trypsinization, and the like. See, J. Bonafaciano, supra, previously incorporated in its entirety.

Once harvested, the cells can be diluted in a second tissue culture medium supplemented with serum and a cryoprotectant. The culture medium can be the same culture medium as the first culture medium, or it can be a different culture media from the first culture medium. Preferably, culture media specific for the eukaryotic cell type or cell line can be used as the base for the second culture medium. The second culture medium can be supplemented with the same type of serum used to supplement the first culture medium, or it can be a different type of serum from the first culture medium. Preferably, fetal bovine serum can be used to supplement the second culture medium. The second culture medium can be supplemented with a final concentration of serum that is greater than the final concentration of serum in the first culture medium. In some embodiments, the final concentration of serum can be between 10% and 40% (v/v). For example, the second culture medium can be supplemented with about 10%, 12%, 14%, 16%, 18%, 20%, 21%, 22%, 23%, 24%, 25%, 26%, 27%, 28%, 29%, 30%, 31%, 32%, 33%, 34%, 35%, 36%, 37%, 38%, 39%, 40% (v/v) serum, or any percentage in between. Preferably, the second culture medium is supplemented with between about 25% and about 35% (v/v), most preferably about 30% (v/v) serum. The second culture medium can comprise additional supplements, such as non-essential amino acids, as described above in reference to the first culture medium.

The second culture medium can be supplemented with any cryoprotectant or combination of cryoprotectants known or discovered in the future. As used herein, the term cryoprotectant refers to a substance that protects cells from damage during freezing. Cryoprotectants can be classified as either “permeating cryoprotectants,” that can penetrate the cell membrane and be present intracellularly, or “non permeating cryoprotectants” that do not penetrate the cell membrane and remain the in extracellular solution. Examples of permeating cryoprotectants include dimethyl sulfoxide (DMSO), glycerol, propylene glycol, ethylene glycol, glycerol, and 1,2-propanediol. Permeating cryoprotectants tend to work by depressing the freezing point which lowers the temperature at which ice is formed, and by reducing the amount of ice formed which, in turn, lowers the temperature at which a specific concentration of electrolytes occurs. Examples of non-permeating cryoprotectants include high molecular weight additives, such as sugars, starches, protein, and other macromolecules. These cryoprotectants tend to work by promoting osmotic water loss from cells at higher subzero temperatures but contribute to osmotic stresses on the cell. In preferred embodiments, the second culture medium is supplemented with DMSO.

The cryoprotectant or combination of cryoprotectants can be present in the second culture medium in an amount from about 3% to about 20% (v/v) final concentration. For example, the cryoprotectant can be present in the second culture medium in about 3%, 4%, 5%, 6%, 7%, 8%, 9%, 10%, 11%, 12%, 13%, 14%, 15%, 16%, 17%, 18%, 19%, 20%, (v/v), or any amount in between. Preferably, the cryoprotectant is present at about 5% to about 15% (v/v) final concentration, and most preferably at about 10% (v/v) final concentration.

Optionally, the second culture medium can be supplemented with a sugar or sugar alcohol. Non-limiting examples of sugars and sugar alcohols that can be used to supplement the second culture medium include monosaccharides, disaccharides, or trisaccharides. Non-limiting examples of monosaccharides useful in the embodiments disclosed herein include glucose, fructose, galactose, ribose and the like. Non-limiting examples of disaccharides useful in the embodiments disclosed herein include sucrose, lactose, maltose, trehalose, and cellobiose. Non-limiting examples of trisaccharides useful in the embodiments described herein include raffinose, arcarbose and melezitose and maltotriose. In preferred embodiments, the second culture medium is supplemented with trehalose. Non limiting examples of sugar alcohols useful in the embodiments disclosed herein can include sorbitol, mannitol, maltitol, and xylitol and the like.

The final concentration of the disaccharide in the second culture medium can be between about 0.1% to about 20% (w/v), and is preferably between about 5% and about 15%, most preferably 10%. For example, the second culture medium can include a disaccharide at a final concentration (w/v) of about 0.1%, 0/5%, 1%, 2%, 3%, 4%, 5%, 6%, 7%, 8%, 9%, 10%, 11%, 12%, 13%, 14%, 15%, 16%, 17%, 18%, 19%, 20%, or any amount in between.

The eukaryotic cells can be diluted in the second culture medium to a final concentration of about 1×10⁵ cells/mL to about 1×10⁷ cells/mL, preferably between about 2×10⁵ cells/mL and 7×10⁵ cells/mL, and most preferably at a final concentration of about 5×10⁵ cells/mL. The diluted cells can be aliquoted into a vial. Preferably, the vial is designed to maintain its integrity upon freezing.

The vial(s) or containers containing the diluted eukaryotic cells is then frozen. As used herein, the term “frozen” is used to describe an object, such as a vial of eukaryotic cells, which is subjected to temperatures below about 0° C. Typically, the diluted eukaryotic cells are subjected to temperatures below about 0° C. and stored indefinitely until future use. Optionally, the vials containing the eukaryotic cells can be placed in a freezer set at −5° C., −10° C., −15° C., −20° C., −25° C., −30° C., −35° C., −40° C., −45° C., −50° C., −60° C., −70° C., −80° C., or lower, or placed in liquid nitrogen.

Preferably, the vials are frozen within about 7 hours of diluting the cells in the second culture medium. Most preferably, the vials are frozen immediately after the diluted cells have been aliquoted into the vials. The term “immediately” as used herein can to refer to an amount of time less than 7 hours, less than 6 hours, less than 5 hours, less than 4 hours, less than 3 hours, less 1 hour, less than 50 minutes, less than 45 minutes, less than 40 minutes, less than 35 minutes, less than 30 minutes, less than 25 minutes, less than 20 minutes, less than 15 minutes, less than 10 minutes, less than 5 minutes, less than 4 minutes, less than 3 minutes, less than 2 minutes, less than 1 minute, or any amount of time in between.

Some embodiments relate to transfection ready eukaryotic cells or competent cells. Preferably, the cells are those made by the process described above and elsewhere herein. The cells can be those deposited with American Type Culture Collection and assigned ATCC numbers: CRL-1573 (HEK 293); CCl-2 (HeLa), CRL 1651 (COS-7); CCl-61 (CHO-K1); HTB-22 (MCF-7); TIB-152 (Jurkat); TIB-71 (RAW264), and the like. The cells can have the surprising ability to be thawed and transfected without the need for prolonged culturing before they can be used for transfection.

Some embodiments provided herein relate to kits that comprise cells or vials of frozen, transfection ready eukaryotic cells, such as those described above and elsewhere herein. For example, some embodiments provide a eukaryotic cell transfection kit that includes a frozen population of eukaryotic cells at a concentration of about 5×10⁶ cells/mL in DMEM media supplemented with about 30% serum and a cryoprotectant, wherein said cryoprotectant is present in a concentration of about 10-30%.

Optionally, the eukaryotic cell transfection kit includes instructions for transforming the frozen population of eukaryotic cells. For example, the instructions can describe the use of cells, compositions and kits described herein with the transfection protocols described elsewhere herein, or any other suitable transfection protocol.

The kits can include, in addition to vials of frozen, transfection ready eukaryotic cells, one or more components such as sterile culture media, sterile media supplements, and other reagents used in cell transfection protocols.

### Methods of Using Transfection Ready Eukaryotic Cells

Provided herein are methods for using the transfection ready eukaryotic cells described herein, preferably cells that have been made according to the methods described herein, including methods of transfecting a eukaryotic cell, methods of increasing the transfection efficiency of a eukaryotic cell, methods of increasing the density of cells in a transfection reaction, methods of screening transfected eukaryotic cells and the like.

As used herein, the phrase “transfecting a eukaryotic cell” refers to the procedure of introducing a macromolecule of interest, such as a nucleic acid, protein, or other macromolecule across the cell membrane of the eukaryotic cell and into the cell. In preferred embodiments, the transfection involves the transfer of nucleic acids into the eukaryotic cell. The term “nucleic acids” as used herein can refers to DNA, RNA, cDNA, siRNA, or any plurality of nucleotides or nucleotide analogs that are covalently linked together. Several nucleotide analogs that are used and that can be present in nucleic acids used in the methods described herein can have alternate backbones, comprising, for example, phosphoramide (Beaucage et al., Tetrahedron 49(10): 1925 (1993) and references therein; Letsinger, J. Org. Chem. 35:3800 (1970); Sprinzl et al., Eur. J. Biochem. 81:579 (1977); Letsinger et al., Nucl. Acids Res. 14:3487 (1986); Sawai et al, Chem. Lett. 805 (1984), Letsinger et al., J. Am. Chem. Soc. 110:4470 (1988); and Pauwels et al., Chemica Scripta 26:141 91986)), phosphorothioate (Mag et al., Nucleic Acids Res. 19:1437 (1991); and U.S. Pat. No. 5,644,048), phosphorodithioate (Briu et al., J. Am. Chem. Soc. 111:2321 (1989), O-methylphosphoroamidite linkages (see Eckstein, Oligonucleotides and Analogues: A Practical Approach, Oxford University Press), and peptide nucleic acid backbones and linkages (see Egholm, J. Am. Chem. Soc. 114:1895 (1992); Meier et al., Chem. Int. Ed. Engl. 31:1008 (1992); Nielsen, Nature, 365:566 (1993); Carlsson et al., Nature 380:207 (1996), each of which is incorporated by reference in its entirety). Other analog nucleic acids include those with positively-charged backbones (Denpcy et al., Proc. Natl. Acad. Sci. USA 92:6097 (1995); non-ionic backbones (U.S. Pat. Nos. 5,386,023, 5,637,684, 5,602,240, 5,216,141 and 4,469,863; Kiedrowshi et al., Angew. Chem. Intl. Ed. English 30:423 (1991); Letsinger et al., J. Am. Chem. Soc. 110:4470 (1988); Letsinger et al., Nucleoside & Nucleotide 13:1597 (1994); Chapters 2 and 3, ASC Symposium Series 580, “Carbohydrate Modifications in Antisense Research”, Ed. Y. S. Sanghui and P. Dan Cook; Mesmaeker et al., Bioorganic & Medicinal Chem. Lett. 4:395 (1994); Jeffs et al., J. Biomolecular NMR 34:17 (1994); Tetrahedron Lett. 37:743 (1996)) and non-ribose backbones, including those described in U.S. Pat. Nos. 5,235,033 and 5,034,506, and Chapters 6 and 7, ASC Symposium Series 580, “Carbohydrate Modifications in Antisense Research”, Ed. Y. S. Sanghui and P. Dan Cook. Nucleic acids containing one or more carbocyclic sugars are also included within the definition of nucleic acids (see Jenkins et al., Chem. Soc. Rev. (1995) pp. 69-176). Several nucleic acid analogs are described in Rawls, C & E News Jun. 2, 1997 p. 35. All of these references are hereby expressly incorporated by reference in their entireties.

As discussed above, some embodiments provide methods of increasing the transfection efficiency of a eukaryotic cell. Transfection efficiency refers to the percentage of cells which have acquired the characteristic conferred by the introduced macromolecule, e.g., nucleic acid encoding a gene, or the aggregate amount of the nucleic acid of interest taken-up by the cells as determined by assaying for a gene product encoded in the transfected nucleic acid. Accordingly, an increase in transfection efficiency refers to an increase in the percentage of cells that acquire the macromolecule of interest or an increase in the aggregate amount of the nucleic acid acquired by the cells.

In some embodiments, the methods can include the step of providing a cell prepared as described herein, for example, a frozen eukaryotic cell. As described above, the eukaryotic cell can be any eukaryotic cell, including a yeast cell, insect cell, plant cell, or animal cell, such as a mammalian cell. In preferred embodiments, the eukaryotic cell can be a mammalian cell such as a CHO cell, COS-7 cell, NIH-3T3 cell, HEK 293 cell, HeLa cell, MCF-7 cell, Jurkat cell or RAW 264 cell, or the like.

The eukaryotic cell may be present in medium supplemented with serum and at least one cryoprotectant as described above. For example, in some embodiments the eukaryotic cell is frozen in culture medium such as Dulbecco's Modified Eagle's Medium (DMEM), Minimum Essential Media Eagle (MEM), Medium 199, Ames' Media, BGJb Medium (Fitton-Jackson Modification), Click's Medium, CMRL-1066 Medium, Fischer's Medium, Glascow Minimum Essential Medium (GMEM), Iscove's Modified Dulbecco's Medium (IMDM), L-15 Medium (Leibovitz), McCoy's 5A Modified Medium, NCTC Medium, Swim's S-77 Medium, Waymouth Medium, William's Medium E, RPMI Medium, and the like. Preferably, mammalian cells are cultured in DMEM Medium.

The medium in which the eukaryotic cell is frozen can be supplemented with serum derived from calves, e.g., fetal bovine serum, adult cows, newborn cows, horses, pigs, rabbits, goats, or any other mammal. The serum present in the medium at a concentration of at least about 10% to about 40% (v/v). For example, the culture medium in which the eukaryotic cells are frozen can include about 10%, 12%, 14%, 16%, 18%, 20%, 21%, 22%, 23%, 24%, 25%, 26%, 27%, 28%, 29%, 30%, 31%, 32%, 33%, 34%, 35%, 36%, 37%, 38%, 39%, 40% (v/v) serum, or any percentage in between. Preferably, the medium is supplemented with between about 25% and about 30% (v/v), most preferably about 30% (v/v) serum. The culture medium in which the eukaryotic cells are frozen can comprise additional supplements, such as non-essential amino acids.

The culture medium in which the eukaryotic cells are frozen can also include a cryoprotectant or combinations of cryoprotectants, such as dimethyl sulfoxide (DMSO), glycerol, propylene glycol, ethylene glycol, glycerol, 1,2-propanediol, sugars, starches, protein, and other macromolecules. In preferred embodiments, the culture medium in which the eukaryotic cells are frozen is supplemented with DMSO. The eukaryotic cells can be frozen in culture media with a cryoprotectant or combination of cryoprotectants present in an amount from about 3% to about 20% (v/v) final concentration. For example, the cryoprotectant can be present in the culture medium in about 3%, 4%, 5%, 6%, 7%, 8%, 9%, 10%, 11%, 12%, 13%, 14%, 15%, 16%, 17%, 18%, 19%, 20%, (v/v), or any amount in between. Preferably, the cryoprotectant is present at about 5% to about 15% (v/v) final concentration, and most preferably at about 10% (v/v) final concentration.

The culture medium in which the eukaryotic cells are frozen can also include a sugar or sugar alcohol. In some embodiments, the sugar can be a disaccharide, such as sucrose, lactose, maltose, trehalose, and cellobiose. In some embodiments, the sugar can be a trisaccharide such as raffinose, arcarbose and melezitose and maltotriose. Common sugar alcohols useful in the embodiments disclosed herein include sorbitol, mannitol, maltitol, and xylitol. In preferred embodiments, the eukaryotic cells are frozen in culture medium that is supplemented with trehalose.

In some embodiments, the culture medium in which the eukaryotic cells are frozen can include between about 0.1% to about 20% (w/v), and is preferably between about 5% and about 15%, most preferably 10% (w/v) sugar or sugar alcohol. For example, the second culture medium can include a sugar or sugar alcohol at a final concentration (w/v) of about 0.1%, 0/5%, 1%, 2%, 3%, 4%, 5%, 6%, 7%,8%, 9%, 10%, 11%, 12%, 13%, 14%, 15%, 16%, 17%, 18%, 19%, 20%, or any amount in between.

In some embodiments, the frozen eukaryotic cell has been frozen at a temperature of less than about 0° C., for example at less than about −5° C., −10° C., −15° C., −20° C., −25° C., −30° C., −35° C., −40° C., −45° C., −50° C., −60° C., −70° C., −80° C., or lower, or in liquid nitrogen.

In the methods provided herein, the frozen eukaryotic cell can be thawed, contacted with the macromolecule to be transfected into the cell within less than five hours from the time the frozen cell is provided. As used herein, the term “thaw” or “thawed” refers to the process of raising the temperature of the object to be thawed, such as frozen eukaryotic cells, to greater than 0° C., and preferably to between about 25° C. and 42° C., most preferably to between about 30° C. and 40° C., e.g., to about 37° C. The frozen eukaryotic cell can be thawed by placing the cells in a water bath set to the desired temperature, e.g., 37° C., by placing the cells on wet ice, leaving the cells at room temperature, or any combination of the procedures. Preferably, the frozen eukaryotic cells are thawed by placing the cells in a 37° C. water bath.

The thawed cells can then be contacted with the macromolecule of interest, e.g., a nucleic acid, within less than about seven hours from the time the cells were initially provided. In some embodiments, the thawed eukaryotic cells are contacted with the macromolecule of interest in about 6 hours, 5.75 hours, 5.5 hours, 5.25 hours, 5 hours, 4.75 hours, 4.5 hours, 4.25 hours, 4 hours, 3.75 hours, 3.5 hours, 3.25 hours, 3 hours, 2.75 hours, 2.5 hours, 2.25 hours, 2 hours, 1.75 hours, 1.5 hours, 1.25 hours, 1 hour, 45 minutes, 30 minutes, 15 minutes, or less, or any amount of time in between, after the cells are first provided. Preferably, the cells are contacted with the macromolecule of interest within about 3 hours of providing the frozen eukaryotic cells. As such, some embodiments provide improvements in methods of transfecting eukaryotic cells, wherein the improvement is the elimination of the need to passage the population of eukaryotic cells and grow them to confluency just prior to transfection.

Optionally, the thawed cells can be diluted in fresh culture medium prior to contacting the cells with the macromolecule of interest. In some embodiments, the thawed cells are present in culture media in a density of about 1.2×10⁵ to about 4×10⁵ cells/mL prior to contacting the thawed cells with the macromolecule of interest. The thawed cells can be contacted with the macromolecule of interest in the presence of other reagents or compounds and under conditions to facilitate the transfection of the macromolecules of interest into the cell, such as cationic lipids for lipofection, calcium phosphate salts, electroporation buffers and media, and the like. In some embodiments, the thawed cells can be contacted with the macromolecule of interest and transfected according to any transfection method now known or discovered in the future. Several routine protocols for the transfection of eukaryotic cells are described in Bonafaciano et al., supra.

In some embodiments, routine transfection procedures are carried out on the thawed eukaryotic cells at a density that is greater than the density typically used in the transfection of cells that have not been frozen as described herein, for example, on eukaryotic cells that have been routinely passaged in continuous cell culture. In some embodiments, routine transfection procedures are carried out on a density of thawed eukaryotic cells that is about 1.2 to about 6 times greater than the density of passaged eukaryotic cells prescribed in the transfection procedures. For example, in some embodiments, the thawed eukaryotic cells can be contacted with macromolecules at a density about 3 to 5, preferably about 4 times greater than the cell density of passaged cells using the same transfection protocols. In some embodiments, wherein said thawed eukaryotic cell is contacted with a macromolecule of interest, e.g. a nucleic acid, in the presence of reagents to facilitate lipofection, the thawed eukaryotic cells can be present in a density of about 1.2×10⁵ to about 4×10⁵ cells/mL, for example, at a density of about 3.5×10⁵ cells/mL to about 4×10⁵ cells/mL. Accordingly, some embodiments provide an improved method of transfecting a population of eukaryotic cells, wherein the improvement comprises increasing the density of cells in a transfection reaction.

Also provided herein are methods of screening eukaryotic cells for a detectable signal encoded on a nucleic acid of interest. Detectable signals can include signals that fluoresce or luminesce, are radioactive, or produce an enzyme whose product can be quantified, and the like. In some embodiments, the nucleic acid encodes an enzyme which can act on suitable substrate to generate a luminescent signal, such as for example, luciferase. Synthetic luminescent substrates for these enzymes are well known in the art and are commercially available from companies, such as Tropix Inc. (Bedford, Mass., USA). In some embodiments, the nucleic acid can encode an enzyme capable of acting on a suitable substrate to transform chemical energy into light energy, such as a luciferase. Luciferases proteins catalyze an energy-yielding chemical reaction in which a specific biochemical substance, a luciferin (a naturally occurring substrate), is oxidized by an enzyme having a luciferase activity. Examples of bioluminescent proteins with luciferase activity may be found in U.S. Pat. Nos. 5,229,285, 5,219,737, 5,843,746, 5,196,524, 5,670,356; each of which is incorporated herein by reference in its entirety. Substrates for luciferases include coelenterazine, benzothiazole, luciferin, enol formate, terpene, and aldehyde, and the like. Non-limiting examples of enzymes whose products are quantifiable are β-galactosidase and alkaline phosphatase.

In some embodiments, the nucleic acid can encode a protein that fluoresces when excited with an appropriate electromagnetic radiation. Non-limiting examples proteins that fluoresce when excited include Green Fluorescent Protein (GFP), or isoforms and derivatives thereof such as YFP, EGFP, EYFP and the like (R. Y. Tsien, (1998) Ann. Rev. Biochem. 63:509-544; U.S. Patent Application No. 20050026234); each of which is incorporated herein by reference in its entirety. Red fluorescent protein (RFP) such as a Discosoma RFP; or a fluorescent protein related to an RFP, such as a cyan fluorescent protein (CFP), an enhanced CFP, citrine. Preferably, the positioning of the FDM domain within the FDM-second protein fusion does not alter the activity of the native protein.

In some embodiments, the methods of screening can include the steps of providing a population of frozen eukaryotic cells and thawing the eukaryotic cells as described above.

The thawed eukaryotic cells can be placed in a containment device or vessel. For example, the containment device can be a multiwell assay plate, such as a 24 well, 96 well, 384 well, 1052 well plate, and the like. In some embodiments, the containment device of vessel can be a single tissue culture dish. The thawed cells can be placed in the containment device and transfected with the nucleic acid of interest within less than five hours of placing the thawed cells in the containment device, for example, 5 hours, 4.75 hours, 4.5 hours, 4.25 hours, 4 hours, 3.75 hours, 3.5 hours, 3.25 hours, 3 hours, 2.75 hours, 2.5 hours, 2.25 hours, 2 hours, 1.75 hours, 1.5 hours, 1.25 hours, 1 hour, 45 minutes, 30 minutes, 15 minutes or less after placing the thawed cells in the containment device.

The cells can be transfected using any suitable transfection method, including those now known or discovered in the future, as described above. The transfected cells can be assayed to detect and measure the detectable signal from transfected eukaryotic cells using methods known to those skilled in the art.

While particular embodiments of the invention have been described in detail, it will be apparent to those skilled in the art that these embodiments are exemplary rather than limiting. The following are non-limiting examples of some of the embodiments.

### EXAMPLES

### Example 1

### Methods of Making Frozen Transfection Ready Cells that are Viable and Exhibit Similar Morphology to Passaged Cells

The following experiments were done to determine whether the methods described herein for the preparation of transfection ready eukaryotic cells affect cell viability or morphological characteristics.

A frozen vial of HeLa, HEK 293, or CHO-K1 cells (approximately 1.8×10⁶ cells/vial) was thawed in a water bath at 37° C. Cells were cultured in a 50 mLs DMEM culture media (Gibco Cat. No. 26140-079) supplemented with 10% FBS and 1% non-essential amino acids (NEAA) (Gibco Cat. No. 11140-050). The cultures were incubated 37° C./5% CO₂ until the cells were approximately 100% confluent. The cultures were washed two times with DPBS (Gibco Cat. No. 14190-250). Cells were detached from the culture dish with 2 mL DETACHIN™ cell detachment reagent (Genlantis Cat. No. T100100) according to the manufacturer's instructions. The cell suspensions were split into two Corning Cellstack™ 1-stack culture dishes (Corning Cat. No. 3268), each containing 250 mL DMEM culture media supplemented with 10% FBS and 1% NEAA and grown at 37° C./5% CO₂ until the cells were approximately 90%-100% confluent.

Cells were washed twice with DPS and treated with 12 mL DETACHIN™ cell detachment reagent. Cells were resuspended in 48 mLs DMEM culture media supplemented with 10% FBS and 1% NEAA. 30 mLs of the cell suspensions were transferred to each of two 50 mL conical tubes and centrifuged at 25000 rpm for 10 minutes. The cells were combined and resuspended in 4 mL complete media in a conical flask. DMEM culture media supplemented with 10% FBS and 1% NEAA was added to the cell suspension to bring the total volume to 11 mLs. 5 mLs of the cell suspension was transferred to 1 L Nalgene receiver bottles, and 1 L DMEM culture media supplemented with 10% FBS and 1% NEAA was added. The 1 L cell culture was transferred to a Corning Cellstack™ 5-stack culture dishes (Corning Cat. No. 3319). 250 mL of additional 1 L DMEM culture media supplemented with 10% FBS and 1% NEAA was added to the 5-stack culture. Cells were grown at 37° C./5% CO₂ until the cells were approximately 90%-100% confluent.

Once the cell cultures reached 90%-100% confluency, the cells were washed twice with DPBS. 60 mLs DETACHIN™ cell detachment reagent was added to the culture dish. 250 mL DMEM culture media supplemented with 10% FBS and 1% NEAA was added to the culture dish. The cell suspension was transferred to a 500 mL centrifuge bottle, and the cells were centrifuged at 2500 rpms for 10 minutes. The cell pellets were resuspended in 3 mL freezing medium. The freezing medium was DMEM culture media supplemented with 30% FBS and 10% DMSO. Freezing media was added to a volume of 10 mLs. The resuspended cell pellets from two cultures were combined (20 mLs total) and the volume was brought to 50 mLs with freezing media.

A sample of the cells was removed and assessed for viability using Tryptan Blue. The remainder of the cells were diluted to 5×10⁶ cells/mL. 500 μL aliquots were transferred to 1.5 mL cryotubes (Axygen Cat. No. SCT-150-SS-A-S). The tubes were placed and stored in a −80° C. freezer for future use.

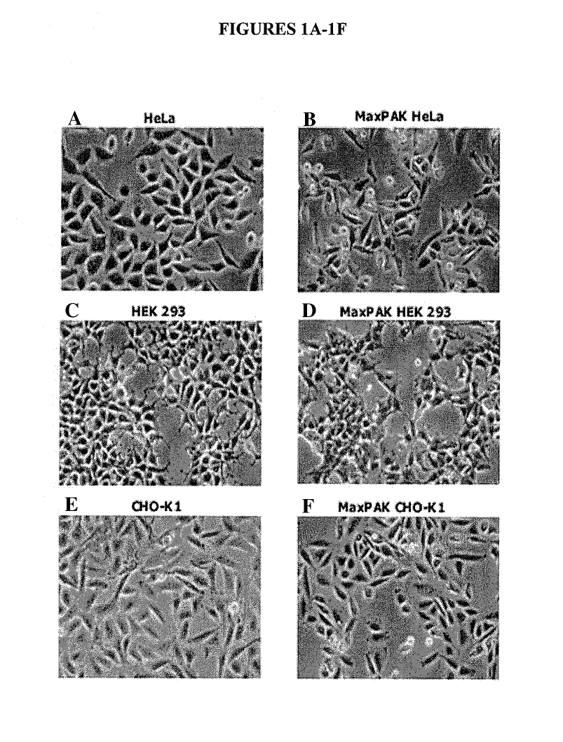

A sample of the frozen cells (transfection ready cells) was quick thawed in a 37° C. water bath and grown for 24 hours in complete DMEM media. The cryopreserved cells were compared to a sample of counterpart cells that had been passaged, but not cryopreserved according to the protocol above. The cell morphology was assessed under a light microscope. The results are shown in FIGS. 1A-1F. The previously cryopreserved cells (HeLa, HEK 293, and CHO-K1) have the same overall cell morphology as the corresponding cell line that was grown under continuous culture (passaged).

### Example 2

### Frozen Transfection Ready Cells can be Plated at Higher Cell Densities than Passaged Cells

Vials of HeLa, HEK 293 and CHO-K1 transfection ready cells made and stored according to the methods described in Example 1 were thawed in a 37° C. water bath. The media was decanted from the cryotube, and 1 mL fresh DMEM media supplemented with 10% FCS was added. 90 μL of the cell suspension was added to 410 μL of fresh DMEM with 10% FCS, transferred to a single well in a 24-well plate and incubated at 37° C./5% CO₂ for 3-4 hours. The cells were compared to non frozen transfected counterpart cells that had been passaged using standard protocols and plated at 40,000 cells/well.

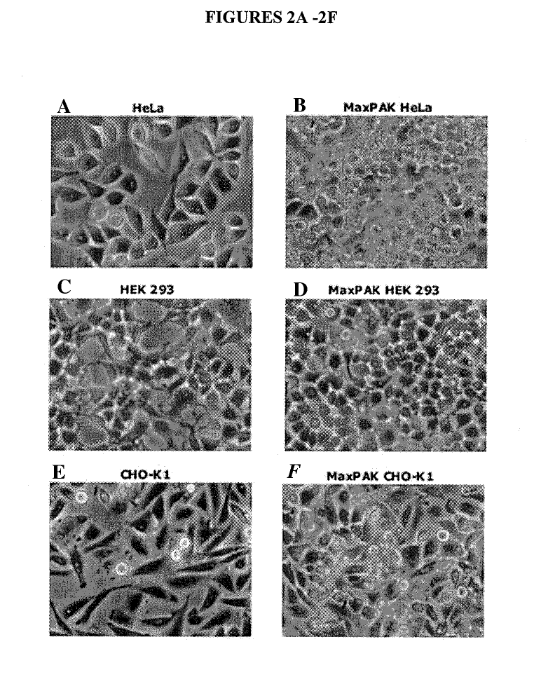

The cells were grown to about 90% confluency and analyzed under a light microscope. The results are presented in FIGS. 2A-2F. The cell density of HeLa, HEK 293 and CHO-K1 cells frozen according to the protocol in Example 1 (FIGS. 1B, 1D, and 1F, respectively), was much greater than the cell density of the counterpart cells that had not been treated according to Example 1, but that had been grown in continuous culture (passaged) according to standard protocols (FIGS. 1A, 1C, and 1E). This demonstrates that the frozen competent cells can be plated at a higher density than untreated cells. Accordingly, the frozen competent cells are advantageous for screening assays, including but not limited to, high throughput screening and testing of expression constructs, where increased protein yield enhances assay sensitivity.

### Example 3

### Transfection Efficiency in Frozen Transfection-Ready Cells is the Same or Better than Passaged Cells

Frozen competent CHO-K1, HEK 293 and HeLa cells were prepared as described in Example 1. A vial of each was quick thawed in a 37° C. water bath. The media was decanted from the cryotube, and 1 mL fresh DMEM media supplemented with 10% FCS was added. 904, of the cell suspension was added to 410 μL of fresh DMEM with 10% FCS, transferred to a single well in a 24-well plate and incubated at 37° C./5% CO₂ for 3-4 hours.

The cells in each well were transfected with 0.8 μg gWIZ™ mammalian high-expression β-galactosidase vector (Genlantis, San Diego, Calif.) using LIPOFECTAMINE™ 2000 transfection reagent (InVitrogen, San Diego, Calif.), according to the manufacturer's protocols.

Passaged CHO-K1, HEK 293 and HeLa cells were plated at a density of about 1×10⁵ cells/well in a 24 well plate and grown to 80%-95% confluency at 37° C./5% CO₂. The cells were transfected with 0.8 μg gWIZ™ mammalian high-expression β-galactosidase vector (Genlantis, San Diego, Calif.) using LIPOFECTAMINE™ 2000 transfection reagent (InVitrogen, San Diego, Calif.), according to the manufacturer's protocols.

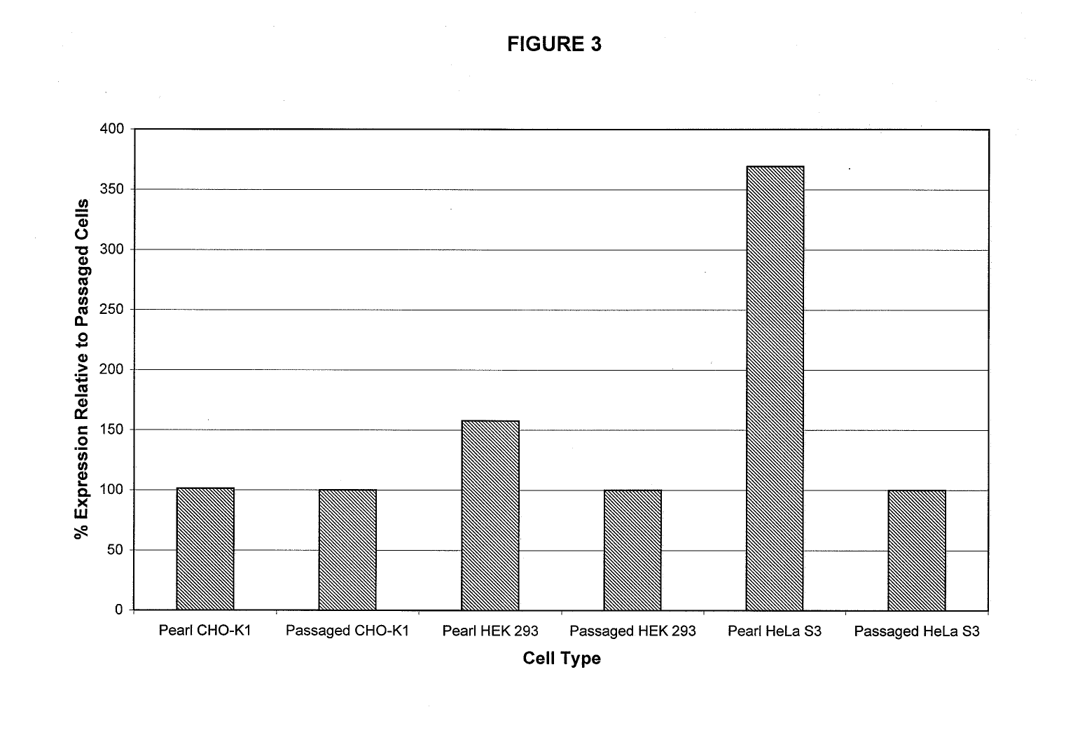

β-galactosidase activity was measured using the X-gal staining kit (Genlantis, San Diego, Calif.) according to the manufacturer's protocols. The measurement of β-galactosidase is proportional to and indicative of the number of plasmid molecules taken up by the population of cells and was determined for both the passaged and the frozen competent cells. The results are presented in FIG. 3. The β-galactosidase activity of CHO-K1 frozen competent cells is about the same as the β-galactosidase activity for passaged cells. The activity of the reporter gene of the frozen competent cells compared to the passaged counterpart varied depending on the cell line; for HEK 293 and HeLa cells, it was substantially greater than the activity of these same cell lines which have not been treated as described in Example 1, i.e., 150% and 350% or greater, respectively.

### Example 4

### Additional Cryoprotectants Increase Transfection Efficiency of Transfection-Ready Cells

Frozen competent HEK 293 cells were prepared as described in Example 1, except that the freezing media comprised DMEM supplemented with 30% FBS, 10% DMSO and 10% trehalose.

A vial of each was quick thawed in a 37° C. water bath. The media was decanted from the cryotube, and 1 mL fresh DMEM media supplemented with 10% FCS was added. 90 μL of the cell suspension was added to 410 μL of fresh DMEM with 10% FCS, transferred to a single well in a 24-well plate and incubated at 37° C./5% CO₂ for 3-4 hours.

The cells in each well were transfected with 0.1, 0.25, 0.5, 0.75 or 1 μg gWIZ™ mammalian high-expression β-galactosidase vector (Genlantis, San Diego, Calif.) using LIPOFECTAMINE™ 2000 transfection reagent (InVitrogen, San Diego, Calif.), according to the manufacturer's protocols.

Passaged HEK 293 were plated at a density of about 1×10⁵ cells/well in a 24 well plate and grown to 80%-95% confluency at 37° C./5% CO₂. The cells were transfected with 0.1, 0.25, 0.5, 0.75 or 1 μg gWIZ™ mammalian high-expression β-galactosidase vector (Genlantis, San Diego, Calif.) using LIPOFECTAMINE™ 2000 transfection reagent (InVitrogen, San Diego, Calif.), according to the manufacturer's protocols.

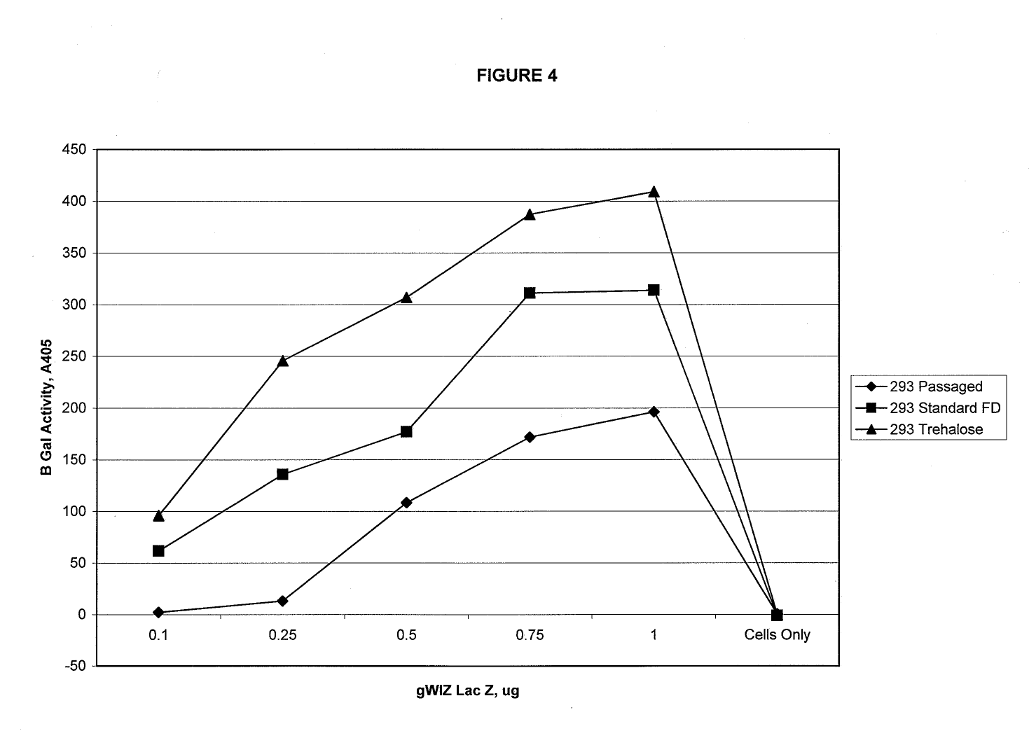

After 48 hours, the transfected cultures were lysed using CelLytic™ cell lysis reagent (Sigma Aldrich, St. Louis, Mo.) and assayed for β-galactosidase activity using the X-gal staining kit (Genlantis, San Diego, Calif.) according to the manufacturer's protocols. The results are presented in FIG. 4. For every concentration of DNA tested, passaged HEK 293 cells produced the weakest β-galactosidase assay signal, demonstrating that the frozen competent cells can provide superior assay sensitivity. Furthermore, for every concentration of DNA tested, cells that were frozen in freezing media with 10% trehalose produced a stronger β-galactosidase assay signal compared to competent cells frozen in media without the disaccharide. This demonstrates that disaccharides can further improve transfection results of frozen competent cells.

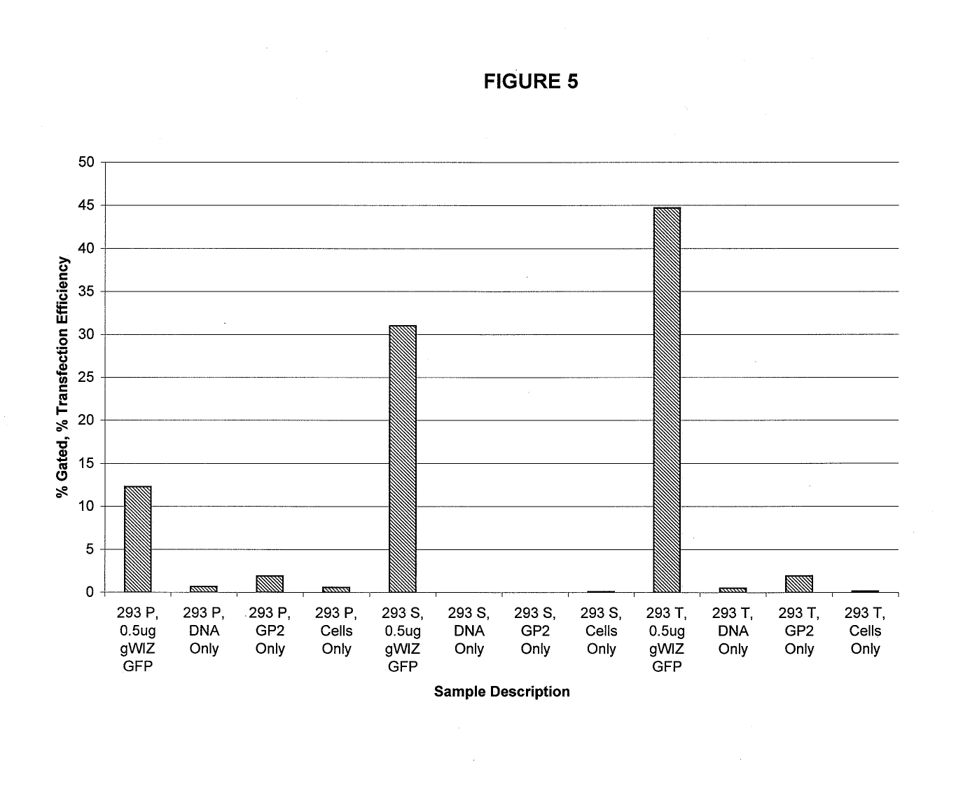

In a separate but similar experiment, passaged HEK 293 cells, competent HEK 293 cells frozen in media without disaccharide, and competent HEK 293 cells frozen in media with disaccharide were transfected with 0.5 μg gWIZ™ mammalian high-expression β-galactosidase vector (Genlantis, San Diego, Calif.) using LIPOFECTAMINE™ 2000 transfection reagent (InVitrogen, San Diego, Calif.), according to the manufacturer's protocols. After 48 hours the cells were analyzed using Fluorescence Activated Cell Sorting (FACS) using standard procedures. Experimental controls for the FACS analysis included DNA only, LIPOFECTAMINE™ 2000 transfection reagent only, and untreated (untransfected) cells only. The results are presented in FIG. 5. The results from this experiment confirm that the transfection efficiency of HEK 293 cells that are frozen competent (with or without disaccharide) is greater than passaged HEK 293 cells. Further, the presence of 10% trehalose in the freezing media increased the transfection efficiency of the frozen HEK 293 competent cells.

The methods, compositions, and devices described herein are presently representative of preferred embodiments and are exemplary and are not intended as limitations on the scope of the invention. Changes therein and other uses will occur to those skilled in the art which are encompassed within the spirit of the invention and are defined by the scope of the disclosure. Accordingly, it will be apparent to one skilled in the art that varying substitutions and modifications can be made to the invention disclosed herein without departing from the scope and spirit of the invention.

As used in the claims below and throughout this disclosure, by the phrase “consisting essentially of” is meant including any elements listed after the phrase, and limited to other elements that do not interfere with or contribute to the activity or action specified in the disclosure for the listed elements. Thus, the phrase “consisting essentially of” indicates that the listed elements are required or mandatory, but that other elements are optional and may or may not be present depending upon whether or not they affect the activity or action of the listed elements.

Numerous literature and patent references have been cited in the present patent application. Each and every reference that is cited in this patent application is incorporated by reference herein in its entirety.

What is claimed is:

1. A method of transfecting a population of eukaryotic cells, comprising:
providing a population of transfection competent eukaryotic cells produced by:
culturing a population of eukaryotic cells in a first culture medium supplemented with about 10% serum until about 80% to about 100% confluency;
harvesting said population of cultured cells;
diluting said population cells in a second medium comprising a culture media supplemented with about 20% to about 40% (v/v) serum and about 5% to about 20% cryoprotectant (v/v) to a density of about 5×10⁶ cells/mL;
aliquoting said population of cells into a containment device; and
freezing said population of cells in said device;
thawing said population of transfection competent eukaryotic cells;
contacting said thawed population of transfection competent eukaryotic cells with a nucleic acid molecule of interest within less than about five hours from said providing step, thereby introducing said nucleic acid molecule of interest into said transfection competent eukaryotic cells.

2. The method of claim 1, wherein said eukaryotic cell is a mammalian cell.

3. The method of claim 1, wherein said contacting step is performed within about 3 hours of said providing step.

4. The method of claim 1, wherein said thawed population of transfection competent cells is contacted with culture medium prior to contacting said population of transfection competent cells with said nucleic acid molecule.

5. The method of claim 1, wherein said thawed population of transfection competent cells is contacted with culture medium to a density of about 1.2×10⁵ to about 4×10⁵ transfection competent cells/mL.

6. The method of claim 1, wherein said second culture medium comprises about 30% (v/v) serum.

7. The method of claim 1, wherein said second culture medium comprises about 10% (v/v) cryoprotectant.

8. The method of claim 1, wherein said cryoprotectant is selected from the group consisting of dimethyl sulfoxide (DMSO), glycerol, 1,2-propanediol, and any combination thereof.

9. The method of claim 8, wherein said cryoprotectant is DMSO.

10. The method of claim 1, wherein said second culture medium further comprises at least compound selected from the group consisting of a sugar or a sugar alcohol at a concentration of about 5% to about 20% (w/v).

11. The method of claim 10, wherein said concentration of said sugar or sugar alcohol is about 10% (w/v).

12. The method of claim 11, wherein said sugar is a trehalose.

13. The method of claim 2, wherein said mammalian cells are selected from the group consisting of CHO cells, HEK293 cells, COS-7 cells, HeLa cells, NIH3T3 cells, MCF-7 cells, Jurkat cells and RAW264 cells.

14. The method of claim 1, wherein said first culture medium and said second culture medium are Dulbecco's Modified Eagle's Medium (DMEM).

15. The method of claim 1, wherein said first culture medium and said second culture medium comprise about 1% (v/v) non-essential amino acids (NEAA). A plurality of frozen eukaryotic cell containment devices wherein each of said plurality of devices comprises a eukaryotic cell aliquot, and wherein said plurality of devices comprises at least 50 devices, wherein each aliquot comprises eukaryotic cells at a concentration of about 1×10⁴ cells/mL to about 1×10⁸ cells/mL in a culture medium supplemented with about 15% to about 40% serum and a cryoprotectant, wherein said cryoprotectant is present in a concentration of about 5-30%.

16. A method of increasing the number of eukaryotic cells transfected in a transfection reaction, comprising:
providing a population of frozen transfection competent eukaryotic cells produced by;
culturing a population of eukaryotic cells in a first culture medium supplemented with about 10% serum until about 80% to about 100% confluency;
harvesting said population of cultured cells;
diluting said population cells in a second medium comprising a culture media supplemented with about 20% to about 40% (v/v) serum and about 5% to about 20% cryoprotectant (v/v) to a density of about 5×10⁶ cells/mL;
aliquoting said population of cells into a containment device; and
freezing said population of cells in said device;
thawing said population of frozen transfection competent eukaryotic cells;
contacting said thawed population of transfection competent eukaryotic cells with culture media to a density of about 1.2×10⁵ to about 4×10⁵ transfection competent cells/mL; and
contacting said transfection competent eukaryotic cell population with a nucleic acid molecule of interest within less than about five hours of said providing step.

17. The method of claim 16, wherein said eukaryotic cells are mammalian cells.

18. The method of claim 16, wherein said transfected eukaryotic cells produce a detectable signal.

19. The method of claim 18, further comprising measuring said detectable signal.

20. The method of claim 16, wherein said mammalian cells are selected from the group consisting of CHO cells, HEK293 cells, COS-7 cells, HeLa cells, NIH3T3 cells, MCF-7 cells, Jurkat cells and RAW264 cells.
